(19) United States
(12) Patent Application Publication
(10) Pub. No.: US 2025/0002981 A1
(43) Pub. Date: Jan. 2, 2025

(54) REAGENTS AND DETECTION METHODS FOR FLUORESCENCE IN SITU HYBRIDIZATION

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Zijian ZHAO, Beijing (CN); Ding DING, Beijing (CN); Shinying LAU, Beijing (CN)

(21) Appl. No.: 18/263,456

(22) PCT Filed: Apr. 29, 2022
(86) PCT No.: PCT/CN2022/090260

§ 371 Date: Jul. 28, 2023

(51) Int. Cl. C12Q 1/6841

(52) U.S. Cl. CPC C12Q 1/6841

(57) ABSTRACT

The present disclosure provides reagents such as a hybridization solution and a pre-digestion treatment solution and kits for fluorescence in situ hybridization, and also provides a detection method for fluorescence in situ hybridization using the above reagents or kits. The fluorescence in situ hybridization can be achieved with ultrafast speed and high resolution.

### FIELD OF THE INVENTION

The present disclosure relates to reagents and detection methods for fluorescence in situ hybridization.

### BACKGROUND OF THE INVENTION

Fluorescence in situ hybridization (FISH) is an important non-radioactive in situ hybridization technique, which appeared in the genetics experiment technique in the late 1970s. According to the principle of complementary base pairing, FISH uses special means to hybridize nucleic acid probes with fluorescent substances to the nucleic acid sequence in the sample to be tested, and finally, the location of the target nucleic acid can be directly observed through a fluorescence microscope. In principle, fluorescence in situ hybridization technology is that if the target DNA on the detected chromosome or DNA fiber section is homologous and complementary to the nucleic acid probe used, the two can be denatured-annealed-refolded to form a hybrid of target DNA and nucleic acid probe. A certain nucleotide of the nucleic acid probe is labeled with a reporter molecule such as biotin or digoxin, and the immunochemical reaction between the reporter molecule and the specific avidin labeled with fluorescein can be used to conduct qualitative, quantitative or relative positioning analysis of the DNA to be tested under a microscope through a fluorescent detection system.

FISH, as the most mature molecular diagnostic technique widely accepted by pathology departments, has a wide range of applications. Gene mapping is the most basic and successful application of fluorescence in situ hybridization. Utilizing sensitiveness, accuracy and ability to detect multiple genes at one time of fluorescence in situ hybridization, the exact position of the target gene can be determined. Determining the positional relationship between several genes, as well as the relationship between genes and chromosomal telomeres, and the relationship between genes and centromeres, is the basic element for constructing a gene map. Fluorescence in situ hybridization is widely used in the physical location of genes and the drawing of gene maps. Its application in tumors has the greatest clinical significance. In solid tumors, it can guide the use of targeted drugs, such as Herceptin (HER2) in breast cancer and crizotinib (ALK, ROS1, CMET) in lung cancer, and it also has great significance in tumor prognosis. Second, the application value in hematological tumors is even greater. In the pathology department, it is used as the gold standard for diagnostic typing, for example, the definite diagnosis of subtypes in lymphoid hematopoietic tumors such as acute promyelocytic leukemia, chronic myeloid leukemia, mantle cell lymphoma, double hit and triple hit lymphoma, and the patient stratification of acute lymphoma, chronic lymphoma, multiple myeloma, etc. In addition, it has a good detection effect on cryptic chromosomal translocations and abnormalities that cannot be found by cytogenetics.

As a successful example of large-scale clinical promotion of modem molecular diagnostic technology, FISH technology is still one of the main technical tools in the pathology departments of major hospitals. The samples submitted for FISH cover a wide range, including tissues, exfoliated cells, amniotic fluid, blood, bone marrow, etc. Moreover, unlike most techniques, FISH samples are not limited to fresh samples, and paraffin-embedded samples can also be tested. At present, the biggest pain point in its clinical practice is that the detection cycle is too long, and shortening the digestion and hybridization time will lead to a significant decrease in its resolution. At present, major leading molecular diagnostic companies (such as Abbott, Roche, etc.) are focusing on the development of fast and high-resolution fluorescent in situ hybridization reagent systems, and the first to make a breakthrough will become the market leader.

### SUMMARY OF THE INVENTION

One aspect of the present disclosure provides reagents or kits for fluorescence in situ hybridization. The reagents include hybridization solution, dewaxing agent, pre-digestion treatment solution, digestion reaction solution, washing working solution, counterstaining solution and fixative solution. The kit includes one or more of the aforementioned reagents.

In an embodiment, the hybridization solution comprises EDTA, PEG, Cot-1 DNA, formamide and polyvinylpyrrolidone (PVP). In a preferred embodiment, the hybridization solution comprises NaCl, sodium citrate, EDTA, PEG, Cot-1 DNA, formamide, polyvinylpyrrolidone (PVP) and anionic surfactant. For example, the hybridization solution comprises 0.5-0.7M of NaCl, 60-80 mM of sodium citrate, 0.1-0.3 mM of EDTA, 0.5-1.0% of PEG, 0.01-0.5% of Cot-1 DNA, 30%-50% of formamide, 0.1-0.2% of PVP and 0.1-1% of anionic surfactant.

In an embodiment, the hybridization solution further comprises polysucrose and/or dextran sulfate, for example 0.01-0.1% of polysucrose and/or 3-15% of dextran sulfate, preferably 0.01-0.05% of polysucrose and/or 8-12% of dextran sulfate.

In an embodiment, the anionic surfactant is SDS and/or lauroyl sarcosine, preferably 0.3-0.5% of SDS and/or 0.1-0.5% of lauroyl sarcosine.

In an embodiment, the hybridization solution further comprises a sodium phosphate or potassium phosphate buffer at pH 6.2-8.0, preferably at pH 6.4-6.8, more preferably at pH 6.6.

In an embodiment, the PEG is a high molecular weight PEG, preferably PEG4000, PEG6000, or PEG8000.

In an embodiment, the hybridization solution further comprises 0.5-2.5 mg/ml of BSA, preferably 1.0-1.5 mg/ml of BSA.

In an embodiment, the hybridization solution comprises 0.5-0.7M of NaCl, 60-80 mM of sodium citrate, 0.1-0.3 mM of EDTA, 0.1-0.2M of sodium phosphate or potassium phosphate buffer (pH 6.4-6.8), 0.05-0.1% of Cot-1 DNA, 0.01-0.05% of polysucrose, 0.1-0.2% of polyvinylpyrrolidone (PVP), 45%-50% of formamide, 10%-12% of dextran sulfate, 0.5-1.0% of PEG4000, 1.2 mg/ml of BSA, and 0.3% of SDS. In another embodiment, the hybridization solution comprises 0.5-0.7M of NaCl, 60-80 mM of sodium citrate, 0.1-0.3 mM of EDTA, 0.1-0.2M of sodium phosphate or potassium phosphate buffer (pH 6.4-6.8), 0.01-0.5% of Cot-1 DNA, 0.1-0.3% of n-lauroyl sarcosine, 0.1-0.2% of polyvinylpyrrolidone (PVP), 30%-33% of formamide, 8-10% of dextran sulfate, 0.5-1.0% of PEG8000, and 0.5% of SDS.

In an embodiment, the dewaxing agent is one or more of xylene, mineral oil, absolute ethanol, isopropanol, and isobutyl laurate.

In an embodiment, the pre-digestion treatment solution comprises Tris, spermine, spermidine, EDTA, EGTA, β-mercaptoethanol, Triton-X and one or more salts selected from KCl, NaCl, and CaCl₂. For example, the pre-digestion treatment solution comprises 5-25 mM of Tris, 0.1-0.5 mM of spermine, 0.1-1.0 mM of spermidine, 1-5 mM of EDTA, 0.2-1.5 mM of EGTA, 40-120 mM of KCl, 10-40 mM of NaCl, 10-20 mM of β-mercaptoethanol, 0.1-0.5% of Triton-X 100, pH 6.9-7.5.

In an embodiment, the digestion reaction solution is a mixed solution of pepsin and hydrochloric acid.

In an embodiment, the washing working solution comprises NaCl, Na₃C₆H₅O₇, and NP-40.

In an embodiment, the counterstaining solution comprises DAPI.

In an embodiment, the fixative solution is one or more of formaldehyde, ethanol, isopropanol, paraformaldehyde, NaH₂PO₄, and Na₂HPO₄.

Another aspect of the present disclosure provides a method for performing fluorescence in situ hybridization using the above kit or one or more of the above reagents.

In an embodiment, the present disclosure provides a detection method for fluorescence in situ hybridization, comprising hybridizing a probe with a sample in the presence of the above hybridization solution, preferably further comprising a step of mixing the sample with the pre-digestion treatment solution before hybridization, wherein the pre-digestion treatment solution comprises Tris, spermine, spermidine, EDTA, EGTA, β-mercaptoethanol, Triton-X and one or more salts selected from KCl, NaCl, and CaCl₂. In a preferred embodiment, the pre-digestion treatment solution comprises 5-25 mM of Tris, 0.1-0.5 mM of spermine, 0.1-1.0 mM of spermidine, 1-5 mM of EDTA, 0.2-1.5 mM of EGTA, 40-120 mM of KCl, 10-40 mM of NaCl, 10-20 mM of β-mercaptoethanol, 0.1-0.5% of Triton-X 100, pH 6.9-7.5.

In an embodiment, the present disclosure provides a detection method for fluorescence in situ hybridization, comprising a step of mixing a sample with a pre-digestion treatment solution before hybridization, wherein the pre-digestion treatment solution comprises Tris, spermine, Spermine, EDTA, EGTA, β-mercaptoethanol, Triton-X and one or more salts selected from KCl, NaCl, and CaCl₂, preferably, the pre-digestion treatment solution comprises 5-25 mM of Tris, 0.1-0.5 mM of spermine, 0.1-1.0 mM of spermidine, 1-5 mM of EDTA, 0.2-1.5 mM of EGTA, 40-120 mM of KCl, 10-40 mM of NaCl, 10-20 mM of β-mercaptoethanol, 0.1-0.5% of Triton-X 100, pH 6.9-7.5.

### BRIEF DESCRIPTION OF THE DRAWINGS

In order to illustrate the technical solutions of the examples of the present invention more clearly, the drawings of the examples will be briefly introduced below. Apparently, the drawings in the following description only relate to some examples of the present invention, rather than limiting the present invention.

FIG. 1 is a fluorescent photograph of a FISH experiment using the reagent of Example 1.

FIG. 2 is a fluorescent photograph of a FISH experiment using the reagent of Example 2.

FIG. 3 is a fluorescent photograph of a FISH experiment using the reagent of Example 3.

FIG. 4 is a fluorescent photograph of a FISH experiment using the reagent of Example 4.

### DETAILED DESCRIPTION OF THE INVENTION

In order to make the purpose, technical solutions and advantages of the examples of the present disclosure more clear, the technical solutions of the examples of the present disclosure will be clearly and completely described below in conjunction with the drawings. Apparently, the described examples are some of the examples of the present disclosure, not all of them. Based on the described examples of the present disclosure, all other examples obtained by those of ordinary skill in the art without creative effort shall fall within the protection scope of the present invention.

Unless otherwise stated, when any type of range is disclosed or claimed, it is intended that each possible value that the range could reasonably encompass is individually disclosed or claimed, including any sub-range encompassed therein. Taking 60-80 mM of sodium citrate as an example, 60-80 should be understood to include the integer values of 60, 61, 62, 63, 64, 65, 66, 67, 68, 69, 70, 71, 72, 73, 74, 75, 76, 77, 78, 79 and 80, decimal values such as 60.1, 60.2, 60.3, 60.4, 60.5, 60.6, 60.7, 60.8 and 60.9, and also sub-ranges such as 61-70, 61.5-75.8 and 60.5-80.

The term “include”, “contain” or “comprise” or a similar term used in the present disclosure means that the element appearing before the term covers the elements listed after the term and their equivalents, and does not exclude unrecited elements. The term “include”, “contain” or “comprise” used herein can be open-ended, semi-closed or closed. In other words, the term also includes “consisting essentially of” or “consisting of”.

Various documents and publications mentioned in this disclosure are hereby incorporated by reference in their entirety into this disclosure. Unless otherwise defined, the technical terms or scientific terms used in the present disclosure shall have the ordinary meanings understood by those skilled in the art to which the present invention belongs. If usage or terminology used in any publications and patents incorporated by reference conflicts with usage or terminology used in the present disclosure, the usage and terminology in the present disclosure shall control.

The first aspect of the present disclosure provides a reagent for rapid fluorescence in situ hybridization in molecular diagnosis, including digestion reaction solution, hybridization solution, washing working solution, counterstaining solution, dewaxing agent, pre-digestion treatment solution, fixative solution, etc., which can effectively shorten the detection time of fluorescence in situ hybridization and is an indispensable key tool in the field of molecular diagnosis, especially in pathological diagnosis, and has great industrial value.

One or more of the dewaxing agent, pre-digestion treatment solution, digestion reaction solution, hybridization solution, washing working solution, counterstaining solution and fixative solution of the present disclosure can be provided as a separate reagent or in the form of a kit.

In an embodiment, the kit includes a digestion reaction solution, a hybridization solution, a washing working solution, and a counterstaining solution. In another embodiment, the kit includes a dewaxing agent, a pre-digestion treatment solution, a digestion reaction solution, a hybridization solution, a washing working solution, and a counterstaining solution. In another embodiment, the kit includes digestion reaction solution, hybridization solution, washing working solution, counterstaining solution, and fixative solution. In another embodiment, the kit includes a dewaxing agent, a pre-digestion treatment solution, a digestion reaction solution, a hybridization solution, a washing working solution, a counterstaining solution and a fixative solution.

For example, the digestion reaction solution is a mixed solution of pepsin and hydrochloric acid, wherein the concentration of pepsin is 0.5 mg/mL to 15 mg/mL, and the pH of hydrochloric acid is controlled to be 3.5-7.0. The purpose is to ensure higher efficiency of subsequent hybridization, which directly determines the permeability.

The whole FISH process includes slide preparation, pretreatment, hybridization, and counterstaining. The hybridization solution is used in the hybridization process. After dehydration and denaturation, the slide is reacted with the hybridization solution in a hybridization box. The hybridization solution is a reagent that allows a probe to be hybridized with the DNA in a nucleus. The hybridization solution comprises NaCl, sodium citrate, ethylenediaminetetraacetic acid (EDTA), polyethylene glycol (PEG), Cot-1 DNA, formamide, polyvinylpyrrolidone (PVP) and anionic surfactant. For example, the hybridization solution comprises 0.5-0.7M of NaCl, 60-80 mM of sodium citrate, 0.1-0.3 mM of EDTA, 0.5-1.0% of PEG, 0.01-0.5% of Cot-1 DNA, 30%-50% of formamide, 0.1-0.2% of PVP and 0.1-1% of anionic surfactant, pH 6.4-8.1. This reagent directly determines the duration of FISH and is the most critical step. Furthermore, this step determines the specificity of probe hybridization and the strength of the background. For example, hybridization can be carried out in a hybridization instrument, with co-denaturation at 75-80° C. for 6-10 min, and incubation at 37° C. for 0.5-1 h. If there is no hybridization instrument, it can be placed on a constant temperature heating plate that has been preheated to 75-80° C. for 6-10 min, then moved into a wet box, and incubated in a 37° C. electric constant temperature incubator for 1 h. For American cytotest and Empire Genomics, the entire hybridization reaction needs to be carried out overnight, that is, for 12-24 h. The hybridization reaction of traditional FISH needs to be carried out overnight, even the hybridization reaction of the rapid products launched by Abbott and Agilent also takes 2-4 hours. However, the hybridization reaction of the present disclosure takes 0.5-1 h, which takes less time, and the hybridization temperature is 37° C., which is significantly lower than the hybridization temperature of 50-70° C. of other products. The hybridization solution of the present disclosure can quickly linearize chromatin, reduce the dissolution temperature, and effectively block non-specific regions, so that the hybridization annealing temperature is lower and the speed is faster.

The PEG is, for example, high molecular weight PEG, preferably one or more selected from PEG4000, PEG6000, and PEG8000.

The Cot-1 DNA refers to a type of DNA with a Cot value equal to 1, wherein the Cot value is a DNA renaturation kinetic parameter. Cot-1 DNA acts as a competitive blocker for nucleic acids, blocking repetitive sequences that may cause non-specific hybridization during hybridization. In a preferred embodiment, the Cot-1 DNA is of human origin, for example commercially available from Roche, 50 to 300 bp in size.

The anionic surfactant is for example selected from SDS and lauroyl sarcosine, preferably 0.1-1% of SDS or 0.05-0.5% of lauroyl sarcosine, more preferably 0.3-0.5% of SDS or 0.1-0.3% of lauroyl sarcosine.

In an embodiment, the hybridization solution further comprises polysucrose and/or dextran sulfate, for example 0.01-0.1% of polysucrose and/or 3-15% of dextran sulfate, preferably 0.01-0.05% of polysucrose and/or 8-12% of dextran sulfate.

In an embodiment, the hybridization solution further comprises a buffer, for example sodium phosphate or potassium phosphate buffer (pH 6.2-8.0, preferably pH 6.4-6.8, more preferably 6.6).

In an embodiment, the hybridization solution further comprises bovine serum albumin (BSA), for example 0.5-2.5 mg/ml of BSA, preferably 1.0-1.5 mg/ml of BSA.

In an embodiment, the hybridization solution comprises 0.5-0.7M of NaCl, 60-80 mM of sodium citrate, 0.1-0.3 mM of EDTA, 0.1-0.2M of sodium phosphate or potassium phosphate buffer (pH 6.4-6.8), 0.05-0.1% of Cot-1 DNA, 0.01-0.05% of polysucrose, 0.1-0.2% of polyvinylpyrrolidone (PVP), 45%-50% of formamide, 10%-12% of dextran sulfate, 0.5-1.0% of PEG4000, 1.2 mg/ml of BSA, and 0.3% of SDS.

In another embodiment, the hybridization solution comprises 0.5-0.7M of NaCl, 60-80 mM of sodium citrate, 0.1-0.3 mM of EDTA, 0.1-0.2M of sodium phosphate or potassium phosphate buffer (pH 6.4-6.8), 0.01-0.5% of Cot-1 DNA, 0.1-0.3% of n-lauroyl sarcosine, 0.1-0.2% of polyvinylpyrrolidone (PVP), 30%-33% of formamide, 8-10% of dextran sulfate, 0.5-1.0% of PEG8000, and 0.5% of SDS.

For example, the washing working solution comprises NaCl, Na₃C₆H₅O₇, and ethylphenyl polyethylene glycol (NP-40). For example, the washing working solution comprises 0.1% of Np-40 or 0.3% of Np-40.

Taking the preparation of 25 L of washing working solution (0.1% of Np-40) as an example, 440 g of sodium chloride and 220 g of sodium citrate are weighed and put into corresponding containers; an appropriate amount of purified water is added, and the mixture is fully stirred until dissolved (gentle heating can accelerate its dissolution); 25 ml of NP-40 is added and mixed thoroughly, during which a large amount of foam would be produced; the pH is adjusted to 7.0±0.2 after the foam dissipates; and the mixture is accurately adjusted to 25 L in a container with a corresponding measuring range, stirred for 5 minutes until uniform, and distributed into 500 ml bottles.

In an embodiment, the counterstaining solution comprises 4′,6-diamidino-2-phenylindole (DAPI). For example, a counterstaining solution can be prepared by dissolving 25 mg of DAPI in 5 ml of purified water.

Formalin-fixed paraffin embedded (FFPE) section samples are postoperative or biopsy histological samples. A sample is fixed in formalin, dehydrated with alcohol, transparentized with xylene, and infiltrated with liquid paraffin to obtain a paraffin block; and the paraffin block is sectioned and then mounted on a glass slide to obtain a paraffin-embedded section. FFPE section samples are the most common sample form in clinical practice, and are important detection objects in pathology departments.

For FFPE section samples, pretreatment (baking, dewaxing, dehydrating, cooking, digesting) is required to digest the paraffin, and the hybridization experiment can be carried out only after sufficient exposure of tissue samples. The steps of hybridization include dehydration, denaturation, hybridization, and washing. The hybridization is the core step of detection, wherein a probe is used to linearize the nucleic acid on the chromatin to a certain extent under an appropriate condition, and hybridization is carried out at an appropriate temperature according to factors such as GC content, so as to characterize the abnormal nucleic acid on the genome. After the hybridization is completed, counterstaining is carried out, so that the nuclei and detection site have clear fluorescent signals for easy observation.

For FFPE section samples, the fluorescence in situ hybridization experiments require sections to be treated sequentially with a dewaxing agent and a pre-digestion treatment solution prior to digestion.

The dewaxing agent is, for example, one or more of xylene, mineral oil, absolute ethanol, isopropanol, and isobutyl laurate. The purpose of a dewaxing agent is to remove the paraffin on a paraffin section and expose a tissue sample.

The pre-digestion treatment solution, for example, comprises Tris, spermine, spermidine, EDTA, EGTA, β-mercaptoethanol, Triton-X and one or more salts selected from KCl, NaCl, and CaCl₂. This reagent is mainly used to increase the permeability of the tissue before digestion, which facilitates the entry of a hybridization probe into tissue cells, increases the probability of contact between the probe and the target nucleic acid, and avoids the loss of nucleic acid due to over-treatment. The volume percentage of Triton-X content can be 0.1%-5%. In an embodiment, the pre-digestion treatment solution comprises 5-25 mM of Tris, 0.1-0.5 mM of spermine, 0.1-1.0 mM of spermidine, 1-5 mM of EDTA, 0.2-1.5 mM of EGTA, 40-120 mM of KCl, 10-40 mM of NaCl, 10-20 mM of β-mercaptoethanol, 0.1-0.5% of Triton-X 100, pH 6.9-7.5. In an embodiment, the pre-digestion treatment solution comprises 5-15 mM of Tris, 0.2-0.5 mM of spermine, 0.1-0.5 mM of spermidine, 1-2 mM of EDTA, 0.5-1.5 mM of EGTA, 80-120 mM of KCl, 20-40 mM of NaCl, 14-20 mM of β-mercaptoethanol, 0.25% of Triton-X 100, pH 7.2±0.2, for example, the pre-digestion treatment solution comprises 15 mM of Tris, 0.2 mM of spermine, 0.5 mM of spermidine, 2 mM of EDTA, 0.5 mM of EGTA, 80 mM of KCl, 20 mM of NaCl, 14 mM of β-mercaptoethanol, 0.25% of Triton-X 100, pH 7.2.

Cell samples include blood cells and various exfoliated cells, including urine, cervical samples, pleural fluid, amniotic fluid, etc., which contain exfoliated cells from various tissues. Cell samples are common in both scientific research and clinical practice. Compared with prepared FFPE, the cell sample requires one more step of slide preparation, including fixing, embedding, and slicing. If the fixation strength of the cell sample is not enough, the signal will be lost, and if the fixation strength of the cell sample is too strong, the cross-linking of macromolecules will be serious and the indigestible background noise will be large. The section thickness of the cell sample is also very relevant. If it is thin, it is easy to be over-digested and the signal will be lost. If it is thick, the indigestible background noise will be large.

In fluorescence in situ hybridization experiments, cell samples need to be treated with a fixative solution prior to hybridization.

The fixative solution is one or more of formaldehyde, ethanol, isopropanol, paraformaldehyde, NaH₂PO₄, and Na₂HPO₄, and is used for fixation in specimen section preparation. It is believed that the fixative solution can avoid signal loss due to excessive cross-linking of macromolecules. The volume percentage of an organic component representing the ratio of an organic component to water phase is 0.5%-50%, preferably 40%, and the molar concentration is 0.1 mol/L-5 mol/L. For example, the fixative solution is 1-4% neutral formaldehyde, 80%-95% ethanol, 4% paraformaldehyde, and ethanol-formaldehyde (AF) fixative solution. The 4% neutral formaldehyde can be prepared by mixing 100 ml of 40% formaldehyde, 6.5 g of anhydrous disodium hydrogen phosphate, 4.0 g of sodium dihydrogen phosphate, and 900 ml of distilled water. The 4% paraformaldehyde can be prepared by mixing 40 g of paraformaldehyde powder, 2.965 g of sodium dihydrogen phosphate, 29 g of anhydrous disodium hydrogen phosphate, and 1000 ml of distilled water. The AF fixative solution can be prepared by mixing 100 ml of 40% formaldehyde and 900 ml of 95% ethanol.

A second aspect of the present disclosure provides a method of performing fluorescence in situ hybridization using the above kit or one or more of the above reagents. A conventional DNA probe is used in the fluorescence in situ hybridization method of the present disclosure.

For FFPE section samples, the fluorescence in situ hybridization method provided by the present disclosure includes: treating a section sequentially with a dewaxing agent and a pre-digestion treatment solution, and then digesting the section with a digestion reaction solution; treating the section with a hybridization solution, and then washing the section with a washing working solution; and counterstaining the section with a counterstaining solution, wherein one or more of the dewaxing agent, pre-digestion treatment solution, digestion reaction solution, hybridization solution, washing working solution and counterstaining solution are selected from the corresponding reagents described in the first aspect of the present disclosure. In an embodiment, the hybridization solution is selected from the hybridization solution described in the first aspect of the present disclosure, while the dewaxing agent, pre-digestion treatment solution, digestion reaction solution, washing working solution and counterstaining solution may or may not use the corresponding reagents described in the first aspect of the present disclosure. In an embodiment, the pre-digestion treatment solution is selected from the pre-digestion treatment solution described in the first aspect of the present disclosure, while the dewaxing agent, hybridization solution, digestion reaction solution, washing working solution and counterstaining solution may or may not use the corresponding reagents described in the first aspect of the present disclosure. In an embodiment, the hybridization solution is selected from the hybridization solution described in the first aspect of the present disclosure, and the pre-digestion treatment solution is selected from the pre-digestion treatment solution described in the first aspect of the present disclosure, while the dewaxing agent, digestion reaction solution, washing working solution and counterstaining solution may or may not use the corresponding reagents described in the first aspect of the present disclosure.

For cell samples, the fluorescence in situ hybridization method provided by the present disclosure includes: treating a section sequentially with a dewaxing agent and a pre-digestion treatment solution, and then digesting the section with a digestion reaction solution; fixing the section with a fixative solution, and then washing the section with a washing working solution; treating the section with a hybridization solution, and then washing the section with a washing working solution; and counterstaining the section with a counterstaining solution, wherein one or more of the dewaxing agent, pre-digestion treatment solution, digestion reaction solution, fixative solution, hybridization solution, washing working solution and counterstaining solution are selected from the corresponding reagents described in the first aspect of the present disclosure. In an embodiment, the hybridization solution is selected from the hybridization solution described in the first aspect of the present disclosure, while the dewaxing agent, pre-digestion treatment solution, digestion reaction solution, fixative solution, washing working solution and counterstaining solution may or may not use the corresponding reagents described in the first aspect of the present disclosure.

Compared with products on the market, the reagent or kit of the present disclosure has the following beneficial effects: (1) the fluorescence in situ hybridization reagent system of the present disclosure can effectively shorten the time period of the entire FISH detection, and has a significant time advantage; (2) the fluorescence in situ hybridization reagent system of the present disclosure has wide applicability, is suitable for various types of probes, and each has high resolution; and (3) compared with similar products, the raw material cost of the formula of the present disclosure is much lower.

The technical solutions of the present invention will be further described below through specific examples. It should be clear to those skilled in the art that the examples are only for helping to understand the present invention, and should not be regarded as specific limitations on the present invention.

The experimental methods used in the following examples are conventional methods unless otherwise specified.

The materials and reagents used in the following examples can be obtained from commercial sources unless otherwise specified.

Unless otherwise specified, nucleic acid herein refers to DNA and RNA molecules.

Unless otherwise specified, “nM” refers to “n mol/L”, “μM” refers to “μmol/L”, and “mM” refers to “m mol/L” herein. The Cot-1 DNA in the reagents was purchased from Roche, and the rest reagents were purchased from sigma.

As a hybridization equipment, Abbott StatSpin ThermoBrite automatic in situ hybridization instrument 5500 was used.

Fluorescence observations were carried out using BX53 fluorescence microscope from OLYMPUS.

### Example 1 Detection of Paraffin Section Samples

The rapid fluorescence in situ hybridization reagent system of the present disclosure can be used for FISH detection of paraffin tissue samples, and the method includes the following steps.

1. The thickness of paraffin sections should not exceed 4 μm. The paraffin sections should be mounted on a special glass slide for in situ hybridization, and tested immediately after being dried by baking, or used for testing within 1 month after being sealed and stored at −20° C.;

2. The sections were baked in a constant temperature oven at 65° C. for 2 h;

3. The sections were immersed in a dewaxing solution for 2 min*2, and dried naturally;

4. One section was stained with hematoxylin-eosin (HE staining), and observed under a light microscope. The lesion tissue area to be detected was selected. This area was marked on the back of another non-HE-stained section by drawing a circle using a glass pen, and the tissue outside the area was scraped off with a blade;

5. The sections were immersed in purified water preheated to 100° C. for 30 min, and then soaked in purified water at room temperature for 3 min;

6. The sections were immersed in a pre-digestion treatment solution and incubated at room temperature for 10 min;

7. The sections were immersed in a digestion reaction solution and incubated in a 37° C. water bath for 5 to 10 minutes. The sections were observed under a microscope until the outline of cells was clear, then transferred into purified water at room temperature, and soaked for 3 minutes;

8. The sections were immersed sequentially in 70%, 85%, and 100% ethanol for 3 min each, and then dried naturally;

9. The probe was allowed to stand at room temperature for 10 minutes, mixed thoroughly, and immediately spun in a mini centrifuge to remove the wall-mounted liquid for later use (Be careful to avoid light when operating); 5-10 μL of probe was added dropwise to the center of the tissue, and covered with a coverslip; the surface of the tissue was immersed in the liquid, and air bubbles were exhausted; the edges of the coverslip were sealed with mounting glue; all subsequent steps should be carried out in the dark as much as possible;

10. The sections were placed in a hybridization instrument, co-denatured at 78° C. for 8 min, and incubated at 37° C. for 0.5-1 h (If there is no hybridization instrument, the sections can be placed on a constant temperature heating plate that has been preheated to 78° C. for 8 min, then moved into a wet box, and incubated in a 37° C. electric constant temperature incubator for 1 h);

11. The mounting glue was carefully removed with ophthalmic forceps;

12. The sections were immersed in a washing working solution at 37° C. and shaken gently to separate the coverslip from the sections;

13. The sections were immersed in a washing working solution at 72° C. for 1 min while being shaken gently;

14. The sections were immersed in a washing working solution at room temperature for 1 min while being shaken gently;

15. The sections were immersed in 75% ethanol for 2 min while being shaken gently, and then dried naturally at room temperature in the dark;

16. 10-20 μL of counterstaining solution sealant was added dropwise to the center of the hybridization area, and covered with a coverslip; the surface of the tissue was immersed in the liquid and air bubbles were exhausted;

17. The results were observed under a fluorescence microscope with appropriate filter modules and magnifications.

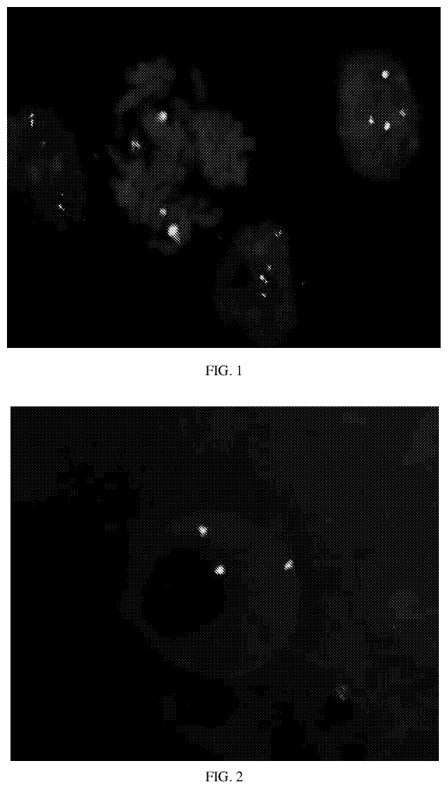

The results are shown in FIG. 1. DAPI stained nuclei. The nuclei were clear, and the fluorescent spots at the detection sites were clear. There was no fluorescent background and no miscellaneous spot, indicating that the results were good.

### Example 2 Detection of Cell Section Samples

The rapid fluorescence in situ hybridization reagent system of the present invention can be used for FISH detection of cell samples, and the method comprises the following steps:
1. Embedding: A wax block was melted into an embedding frame; when the wax block had been cooled but not solidified, the centrifuged cell sample was placed in the wax block and frozen at −20° C.; after solidification, the wax block formed a standard shape wax block;
2. Slicing: The wax block was sliced into sections with a thickness of 4 microns with a microtome, flattened on a slide machine in warm water at 40° C., and transferred onto a glass slide;
3. The cell sections were baked in a constant temperature incubator at 60° C. for 2 hours;
4. The sections were immersed in a dewaxing solution for 2 min*2, and dried naturally;
5. The sections were immersed in a washing working solution at 37° C. for 5 min×2 times;
6. The sections were immersed in purified water preheated to 100° C. for 30 minutes, and then soaked in purified water at room temperature for 3 minutes;
7. The sections were immersed in a pre-digestion treatment solution and incubated at room temperature for 10 minutes;
8. The sections were immersed in a digestion reaction solution at 37° C. for 2-5 minutes, and observed under a microscope until the outline of cells became clear;
9. The sections were immersed in 1% neutral formaldehyde for 5 minutes;
10. The sections were immersed in a washing working solution for 5 min×2 times;
11. The sections were immersed sequentially in 70%, 85%, and 100% ethanol for 3 min each, and then dried naturally;

12. The probe was allowed to stand at room temperature for 10 minutes, mixed thoroughly, and immediately spun in a mini centrifuge to remove the wall-mounted liquid for later use (Be careful to avoid light when operating);
13. 5-10 μL of probe was added dropwise to the center of the tissue, and covered with a coverslip; the surface of the tissue was immersed in the liquid, and air bubbles were exhausted; the edges of the coverslip were sealed with mounting glue; all subsequent steps should be carried out in the dark as much as possible; the sections were placed in a hybridization instrument, co-denatured at 78° C. for 8 min, and incubated at 37° C. for 0.5-1 h (If there is no hybridization instrument, the sections can be placed on a constant temperature heating plate that has been preheated to 78° C. for 8 min, then moved into a wet box, and incubated in a 37° C. electric constant temperature incubator for 1 h);
14. The mounting glue was carefully removed with ophthalmic forceps;
15. The sections were immersed in a washing working solution at 37° C. and shaken gently to separate the coverslip from the sections;
16. The sections were immersed in a washing working solution at 72° C. for 1 min while being shaken gently;
17. The sections were immersed in a washing working solution at room temperature for 1 min while being shaken gently;
18. The sections were immersed in 75% ethanol for 2 min while being shaken gently, and then dried naturally at room temperature in the dark;
19. 10-20 μL of counterstaining solution sealant was added dropwise to the center of the hybridization area, and covered with a coverslip; the surface of the tissue was immersed in the liquid and air bubbles were exhausted;
20. The results were observed under a fluorescence microscope with appropriate filter modules and magnifications.

The results are shown in FIG. 2. DAPI stained nuclei. The nuclei were clear, and the fluorescent spots at the detection sites were clear. There was no fluorescent background and no miscellaneous spot, indicating that the results were good.

### Example 3 Hybridization Solution

0.5-0.7M of NaCl, 60-80 mM of sodium citrate, 0.1-0.3 mM of EDTA, 0.1-0.2M of sodium phosphate or potassium phosphate buffer (pH 6.4-6.8), 0.05-0.1% of Cot-1 DNA, 0.01-0.05% of polysucrose, 0.1-0.2% of polyvinylpyrrolidone (PVP), 45%-50% of formamide, 10%-12% of dextran sulfate, 0.5-1.0% of PEG4000, 1.2 mg/ml of BSA, and 0.3% of SDS.

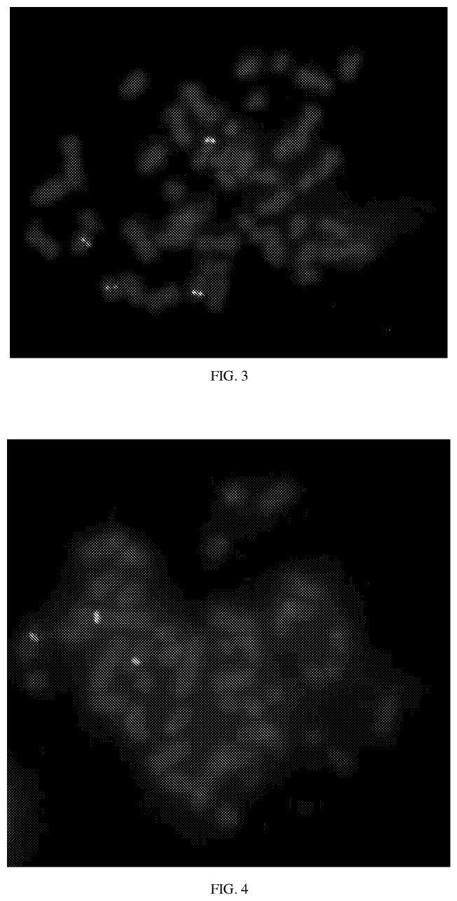

A formula of hybridization solution was provided in Example 3, and a FISH experiment was carried out using the hybridization solution. The results are as shown in FIG. 3. DAPI stained nuclei. The nuclei were clear, and the fluorescent spots at the detection sites were clear. There was no fluorescent background and no miscellaneous spot, indicating that the results were good.

### Example 4 Hybridization Solution 2

0.5-0.7M of NaCl, 60-80 mM of sodium citrate, 0.1-0.3 mM of EDTA, 0.1-0.2M of sodium phosphate or potassium phosphate buffer (pH 6.4-6.8), 0.01-0.5% of Cot-1 DNA, 0.1-0.3% of n-lauroyl sarcosine, 0.1-0.2% of polyvinylpyrrolidone (PVP), 30%-33% of formamide, 8-10% of dextran sulfate, 0.5-1.0% of PEG8000, and 0.5% of SDS.

A formula of hybridization solution was provided in Example 4, and a FISH experiment was carried out using the hybridization solution. The results are as shown in FIG. 4. DAPI stained nuclei. The nuclei were clear, and the fluorescent spots at the detection sites were clear. There was no fluorescent background and no miscellaneous spot, indicating that the results were good.

### Example 5 Pre-Digestion Treatment Solution

15 mM of Tris, 0.2 mM of spermine, 0.5 mM of spermidine, 2 mM of EDTA, 0.5 mM of EGTA, 80 mM of KCl, 20 mM of NaCl, 14 mM of β-mercaptoethanol, 0.25% of Triton-X 100, pH 7.2.

A formula of pre-digestion treatment solution was provided in Example 5.

The present invention illustrates the process method of the present invention through the above examples, but the present invention is not limited to the above process steps, that is, it does not mean that the present invention must rely on the above process steps to be implemented. Those skilled in the art should understand that any improvement of the present invention, the equivalent replacement of the selected raw materials in the present invention, the addition of auxiliary components, the selection of specific manners, etc., all fall within the scope of protection and disclosure of the present invention.

In the case of no conflict, the embodiments of the present invention and the features in the embodiments can be combined with each other to obtain new embodiments. The protection scope of the present invention is not limited to the above embodiments, and the protection scope of the present invention should be controlled by the protection scope of the claims.

1. A hybridization solution for fluorescence in situ hybridization, comprising: EDTA, PEG, Cot-1 DNA, formamide, and polyvinylpyrrolidone (PVP).

2. The hybridization solution according to claim 1, further comprising: NaCl, sodium citrate, and an anionic surfactant.

3. The hybridization solution according to claim 2, comprising: 0.5-0.7M of NaCl, 60-80 mM of sodium citrate, 0.1-0.3 mM of EDTA, 0.5-1.0% of PEG, 0.01-0.5% of Cot-1 DNA, 30%-50% of formamide, 0.1-0.2% of PVP, and 0.1-1% of an anionic surfactant.

4. The hybridization solution according to claim 3, further comprising 0.01-0.1% of polysucrose and/or 3-15% of dextran sulfate.

5. The hybridization solution according to claim 4, wherein the anionic surfactant is SDS and/or lauroyl sarcosine.

6. The hybridization solution according to claim 5, further comprising a sodium phosphate or potassium phosphate buffer at pH 6.2-8.0.

7. The hybridization solution according to claim 6, wherein the PEG is high molecular weight PEG.

8. The hybridization solution according to claim 7, further comprising 0.5-2.5 mg/ml of BSA.

9. The hybridization solution according to claim 1, comprising 0.5-0.7M of NaCl, 60-80 mM of sodium citrate, 0.1-0.3 mM of EDTA, 0.1-0.2M of sodium phosphate or potassium phosphate buffer (pH 6.4-6.8), 0.05-0.1% of Cot-1 DNA, 0.01-0.05% of polysucrose, 0.1-0.2% of polyvinylpyrrolidone (PVP), 45%-50% of formamide, 10%-12% of dextran sulfate, 0.5-1.0% of PEG4000, 1.2 mg/ml of BSA, and 0.3% of SDS; or comprising 0.5-0.7M of NaCl, 60-80 mM of sodium citrate, 0.1-0.3 mM of EDTA, 0.1-0.2M of sodium phosphate or potassium phosphate buffer (pH 6.4-6.8), 0.01-0.5% of Cot-1 DNA, 0.1-0.3% of n-lauroyl sarcosine, 0.1-0.2% of polyvinylpyrrolidone (PVP), 30%-33% of formamide, 8-10% of dextran sulfate, 0.5-1.0% of PEG8000, and 0.5% of SDS.

10. A kit for fluorescence in situ hybridization, comprising the hybridization solution according to claim 1, and dewaxing agent, pre-digestion treatment solution, digestion reaction solution, washing working solution, and counterstaining solution.

11. The kit according to claim 10, wherein the dewaxing agent is one or more of xylene, mineral oil, absolute ethanol, isopropanol, and isobutyl laurate.

12. The kit according to claim 10, wherein the pre-digestion treatment solution comprises Tris, spermine, spermidine, EDTA, EGTA, β-mercaptoethanol, Triton-X and one or more salts selected from KCl, NaCl, and CaCl₂.

13. The kit according to claim 12, wherein the pre-digestion treatment solution comprises 5-25 mM of Tris, 0.1-0.5 mM of spermine, 0.1-1.0 mM of spermidine, 1-5 mM of EDTA, 0.2-1.5 mM of EGTA, 40-120 mM of KCl, 10-40 mM of NaCl, 10-20 mM of (3-mercaptoethanol, 0.1-0.5% of Triton-X 100, pH 6.9-7.5.

14. The kit according to claim 10, wherein the digestion reaction solution is a mixed solution of pepsin and hydrochloric acid, and the washing working solution comprises NaCl, Na₃C₆H₅O₇, and NP-40.

15. (canceled)

16. The kit according to claim 10, wherein the counterstaining solution comprises DAPI, and the kit further comprises a fixative solution which is one or more of formaldehyde, ethanol, isopropanol, paraformaldehyde, NaH₂PO₄, and Na₂HPO₄.

17-18. (canceled)

19. A pre-digestion treatment solution for fluorescence in situ hybridization, comprising Tris, spermine, spermidine, EDTA, EGTA, β-mercaptoethanol, Triton-X, and one or more salts selected from KCl, NaCl, and CaCl₂).

20. The pre-digestion treatment solution according to claim 19, wherein the pre-digestion treatment solution comprises 5-25 mM of Tris, 0.1-0.5 mM of spermine, 0.1-1.0 mM of spermidine, 1-5 mM of EDTA, 0.2-1.5 mM of EGTA, 40-120 mM of KCl, 10-40 mM of NaCl, 10-20 mM of β-mercaptoethanol, 0.1-0.5% of Triton-X 100, pH 6.9-7.5.

21. A detection method for fluorescence in situ hybridization, comprising; mixing a sample with a pre-digestion treatment solution before hybridization, wherein the pre-digestion treatment solution comprises Tris, spermine, spermidine, EDTA, EGTA, β-mercaptoethanol, Triton-X, and one or more salts selected from KCl, NaCl, and CaCl₂); and hybridizing a probe with the sample in the presence of the hybridization solution according to claim 1.

22. (canceled)

23. A detection method for fluorescence in situ hybridization, comprising a step of mixing a sample with a pre-digestion treatment solution before hybridization, wherein the pre-digestion treatment solution comprises Tris, spermine, spermidine, EDTA, EGTA, β-mercaptoethanol, Triton-X, and one or more salts selected from KCl, NaCl, and CaCl₂.

24. The detection method according to claim 23, wherein the pre-digestion treatment solution comprises 5-25 mM of Tris, 0.1-0.5 mM of spermine, 0.1-1.0 mM of spermidine, 1-5 mM of EDTA, 0.2-1.5 mM of EGTA, 40-120 mM of KCl, 10-40 mM of NaCl, 10-20 mM of β-mercaptoethanol, 0.1-0.5% of Triton-X 100, pH 6.9-7.5.
